(19) United States
(12) Patent Application Publication
(10) Pub. No.: US 2025/0327108 A1
(43) Pub. Date: Oct. 23, 2025

(54) Optimized Bioprocessing for Scalable Cell-Free Protein Synthesis

(71) Applicant: Swiftscale Biologics Inc., South San Francisco, CA (US)

(72) Inventors: Danielle Jessica YOESEP, South San Francisco, CA (US); Weston Kevin KIGHTLINGER, South San Francisco, CA (US); Sajita Caroni SHAH-MORALES, South San Francisco, CA (US); Weihua WU, South San Francisco, CA (US)

(21) Appl. No.: 18/855,277

(22) PCT Filed: Apr. 7, 2023
(86) PCT No.: PCT/US2023/017906

§ 371 Date: Oct. 8, 2024

Related U.S. Application Data
(60) Provisional application No. 63/329,295, filed on Apr. 8, 2022.

(51) Int. Cl. C12P 21/00; C12M 1/42

(52) U.S. Cl. CPC C12P 21/00; C12M 35/04

(57) ABSTRACT

Materials, methods, and systems for the bioprocessing of cellular lysates for cell-free protein synthesis are described. For example, a method of improving the bioprocessing efficiency of cell-free protein synthesis comprising preparing a cellular lysate, wherein the prepared cellular lysate has not been clarified, and contacting the prepared cellular lysate with a nucleic acid template encoding a polypeptide template is provided.

### CROSS REFERENCE

This application claims the benefit of PCT Application Serial No. PCT/US2023/017906, filed Apr. 7, 2023, which claims the benefit of U.S. Provisional Application No. 63/329,295, filed Apr. 8, 2022, which are incorporated herein by reference in their entireties.

### BACKGROUND

Cell-free protein synthesis (CFPS) is a platform technology that uses crude cellular extract to synthesize desired protein products rapidly and reliably. This crude cellular extract is the foundation of a cell-free reaction, as it utilizes its endogenous machinery to enable polypeptide translation (and optionally, mRNA transcription) to generate protein products from nucleic acid templates (such as plasmid or linear DNA) as well as supplemented nucleotides, amino acids, an energy source, and other necessary cofactors. CFPS has many potential advantages over cellular expression including faster expression timelines, the freedom to customize expression conditions, and obviating the need to generate unique cell lines for each synthesized protein by direct addition of linear or plasmid DNA templates. However, cell-free extract preparation methods are long and laborious. In order to scale-up CFPS methods and make them feasible for use in a wide variety of settings, cellular extract must be prepared efficiently and inexpensively.

Much effort in the CFPS field has been dedicated to streamlining extract preparation, which is generally composed of cell growth, cell harvest, cell lysis, and post-lysis processing. Post-lysis clarification typically relies on centrifugation. Numerous reports describe attempts to lower the power demands of post-lysis centrifugation step (e.g., lower speed and/or fewer spins). Still, manufacturing-scale protein synthesis requires alternative methods of post-lysis processing that reduce energy costs while maintaining high protein yields.

### SUMMARY

Accordingly, embodiments of the present disclosure are directed to simplified bioprocessing methods of cell-free protein synthesis. The methods described are more efficient, more linearly scalable, and less expensive than current state-of-the-art approaches, as they ultimately decrease the number and power demand of processing steps and therefore the time, equipment, and capital required to produce cell-free extracts and products derived therefrom.

The present disclosure is predicated on the discovery that, against previous teachings, cell-free protein synthesis can be performed using unclarified cellular lysates, while maintaining substantial yields from the cell-free reaction.

A first aspect of the present disclosure includes a method of improving the bioprocessing efficiency of cell-free protein synthesis comprising preparing a cellular lysate, wherein the prepared cellular lysate has not been clarified, and contacting the prepared cellular lysate with a nucleic acid template encoding a polypeptide, and optionally isolating the polypeptide encoded by the nucleic acid template. Preparing the cellular lysate can include harvesting cells of a cellular culture and lysing the cells. The cells can be harvested by subjecting a cellular culture to centrifugation, tangential flow filtration, membrane separation or a combination thereof, optionally membrane separation including hollow fiber membrane separation. The harvested cells can be lysed by sonication, homogenization, nitrogen cavitation, freeze-thaw, syringing, chemical, enzymatic, or osmotic lysis. The harvested cells can be lysed by homogenization at an operating pressure within a range of about 2000 to 30,000 psig, optionally about 5000 to about 30,000 psig. The harvested cells can be lysed at a temperature within a range of about 1° C. to about 30° C., about 1° C. to about 25° C., about 1° C. to about 20° C., or about 1° C. to about 15° C. Contacting the prepared cellular lysate with the nucleic acid template can be performed at a temperature of about 10° C. to about 42° C., about 15° C. to about 37° C., from about 15° C. to about 30° C., or from about 15° C. to about 25° C. The cellular lysate can include lysed cells from a transgenic cell culture, a mammalian cell culture, a bacterial cell culture, plant cell culture, a yeast cell culture, an insect cell culture, a fungal cell culture, or an algal cell culture. The cellular lysate can include lysed cells from an E. coli culture. The cellular lysate can be prepared from a cell culture that is in log or stationary phase. The method can further include a step of culturing cells to an OD₆₀₀ of about 0.5 to about 100, optionally about 5 to about 100 or about 10 to about 100. The method can further include a step of mixing the cellular lysate with one or more components selected from the group consisting of amino acids, nucleotides, salts, cofactors, an energy source, a translation template, and a transcription template, or a combination thereof. The energy source can include a phosphate group or non-phosphorylated energy group. The energy source can be present at a concentration of greater than about 10 mM, 100 mM, 200 mM, 300 mM, but generally less than 400 mM. The energy source can include at least one of phosphoenolpyruvate, glutamate, glycerol, pyruvate, glucose, or creatine phosphate. The energy source can include glutamate. The salt can include potassium at a concentration greater than about 50 mM, 100 mM, 150 mM, 200 mM, 250 mM, 300 mM, 350 mM, 400 mM, or 450 mM, but less than about 500 mM, magnesium at a concentration greater than about 1 mM, 2 mM, 3 mM, 4 mM, 5 mM, 6 mM, 8 mM, 12 mM, 16 mM or 20 mM, but less than about 30 mM, or a combination thereof. The method can further include freeze-drying the cellular lysate. The method can further include freezing the cellular lysate. The method can include isolating the polypeptide encoded by the nucleic acid template. Isolating the polypeptide can be performed at a temperature within a range of about 1° C. to about 30° C., about 1° C. to about 25° C., about 1° C. to about 20° C., or about 1° C. to about 15° C.

In a second aspect, the present disclosure includes a system for continuous bioprocessing for cell-free protein synthesis comprising: a lysing device configured to prepare a cell lysate from cultured cells, wherein the lysing device in fluidic connection with a reaction chamber configured for cell-free protein synthesis, wherein the reaction chamber is configured to receive the prepared cellular lysate and a nucleic acid template encoding a polypeptide. In some cases, the system further includes a fermentation chamber for culturing cells and a cell concentration device for concentrate the cultured cells. The fermentation chamber can be operably connected to the cell concentration device. The cell concentration device can be operably connected to the lysing device. The system can further include a reservoir for components selected from the group consisting of amino acids, nucleotides, salts, cofactors, an energy source, a translation template, and a transcription template, or a combination thereof, wherein the reservoir is in fluidic connection with the reaction chamber. The system can further include a protein isolation device configured to separate synthesized protein from the reaction chamber.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. The drawings illustrate generally, by way of example, but not by way of limitation, various embodiments discussed in the present document.

Reference is made to illustrative embodiments that are depicted in the figures, in which:

FIG. 1 compares the state-of-the-art CFPS methods involving extract clarification via centrifugation (upper) and a method without clarification (lower) according to one or more embodiments of the present disclosure.

FIG. 2 is a bar graph comparing protein, superfolder green fluorescent protein (sfGFP), yield from cell-free protein synthesis reactions using cell-free lysate (left, unclarified) and cell-free extract (right, clarified by centrifugation), according to one or more embodiments of the present disclosure.

### DETAILED DESCRIPTION

Embodiments of the present disclosure describe methods of improving the bioprocessing efficiency of cell-free protein synthesis. For example, methods of cell-free protein synthesis using a cellular lysate for protein synthesis without an intervening clarification step, are provided.

### Definitions

For the present disclosure, terms are defined as follows:

The terms “clarify”, “clarification”, “clarification step,” generally refer to one or more steps used to remove whole cells, cellular debris, and foreign material that is not required for cell-free protein synthesis, including centrifugation and/or filtration.

“Cell lysis” refers to disruption of the cellular membrane. Typically, lysis is carried out in a buffer solution configured to limit or prevent inactivation, denaturation and degradation of cellular proteins. The “cellular lysate” generally includes the contents of lysed cells and the components of the lysis buffer solution. In some cases, the cellular lysate includes foreign material introduced during cell culture. “Cellular lysate” and “cell-free lysate” are used interchangeably in the present disclosure.

“Cell-free extract” refers to a product obtained by clarifying a cellular lysate.

The term “cellular debris” refers to cellular proteins, membranes, cell wall components, and/or genomic DNA typically present in cellular lysates.

Embodiments of the present disclosure describe a method of cell-free protein synthesis (CFPS) using unclarified cellular lysate. Systems configured for and/or adapted to perform one or more steps of the method and reaction mixtures containing unclarified lysates are also described.

### A. Methods

Methods of the present disclosure include cell-free protein synthesis (CFPS) using unclarified cellular lysate. In contrast to methods that prepare a cell extract by clarifying a cellular lysate, the present method utilizes the cellular lysate directly in the CFPS reaction mix to synthesize a protein of interest.

The method of the present disclosure can be used with any cell-free platform. Thus, the cellular lysate to be clarified can be a lysate of any cell culture suitable for cell-free protein synthesis, including bacteria, insect, plant, yeast, fungi, and mammalian cell culture. Non-limiting examples include Escherichia coli, Spodoptera frugiperda, Saccharomyces cerevisiae, Neurospora crassa, Streptomyces, Vibrio natriegens, Bacillus subtilis, Tobacco, Arabidopsis, Pseudomonas Putida, Bacillus megaterium, Archaea, and Leishmania tarentolae, Chinese hamster ovary, rabbit reticulocyte, wheat germ, and HeLa cells.

The cellular lysate can be obtained by any suitable lysis method. Typically, the cells are harvested from the culture media and resuspended prior to lysis. Cells can be resuspended at more than about 10 mL, about 5 mL, about 4 mL, about 3 mL, about 2 mL, about 1 mL, or 0.5 mL per wet weight gram of cells. Any method capable of disrupting cellular membranes can be used. Suitable methods of cell lysis include, for example, french press, bead beating, sonication, homogenization, nitrogen cavitation, freeze-thaw, syringing, and osmotic lysis.

The method of preparing the lysate can be performed at any temperature suitable for culturing, harvesting, or lysing cells in preparation for CFPS. For example, suitable temperature ranges include from about 1° C. to about 35° C., from about 1° C. to about 30° C., and from about 1° C. to about 15° C. In certain aspects, the reaction temperature can be about 1° C., about 5° C., about 10° C., about 15° C., about 20° C., about 25° C., or about 30° C., about 35° C. The lysis temperature can be greater than 0° C., such as about 1° C., about 2° C., about 3° C., about 4° C., about 5° C. about 6° C., about 7° C., about 8° C., about 9° C., about 10° C., about 11° C., about 12° C., about 13° C., about 14° C., about 15° C., about 16° C., about 17° C., about 18° C., about 19° C., or about 20° C.

In some cases, method can include one or more post-lysing steps. For example, the unclarified lysate can be desalted, buffer-exchanged, dialyzed, or diafiltrated based on the properties of the cellular lysate. The post-lysing step can include freezing or freeze-drying the unclarified lysate, or CFPS can be immediately performed after lysis.

The method can include using the lysate in a cell-free protein synthesis method as known in the art. See, for example, U.S. Pat. Nos. 5,478,730; 5,556,769; 5,665,563; 6,168,931; 6,548,276; 6,869,774; 6,994,986; 7,118,883; 7,186,525; 7,189,528; 7,235,382; 7,338,789; 7,387,884; 7,399,610; 7,776,535; 7,817,794; 8,703,471; 8,298,759; 8,715,958; 8,734,856; 8,999,668; and 9,005,920. See also U.S. Published Application Nos. 2018/0016614, 2018/0016612, 2016/0060301, 2015-0259757, 2014/0349353, 2014/0295492, 2014/0255987, 2014/0045267, 2012/0171720, 2008/0138857, 2007/0154983, 2005/0054044, 2004/0209321, 2005/0170452; 2006/0211085; 2006/0234345; 2006/0252672; 2006/0257399; 2006/0286637; 2007/0026485; 2007/0178551. See also Published PCT International Application Nos. 2003/056914; 2004/013151; 2004/035605; 2006/102652; 2006/119987; and 2007/120932. The contents of these patent documents are incorporated in the present application by reference in their entireties. See also Jewett, M. C., Hong, S. H., Kwon, Y. C., Martin, R. W., and Des Soye, B. J. 2014, “Methods for improved in vitro protein synthesis with proteins containing non-standard amino acids,” U.S. Patent Application Ser. No.: 62/044,221; Jewett, M. C., Hodgman, C. E., and Gan, R. 2013, “Methods for yeast cell-free protein synthesis,” U.S. Patent Application Ser. No.: 61/792,290; Jewett, M. C., J. A. Schoborg, and C. E. Hodgman. 2014, “Substrate Replenishment and Byproduct Removal Improve Yeast Cell-Free Protein Synthesis,” U.S. Patent Application Ser. No.: 61/953,275; and Jewett, M. C., Anderson, M. J., Stark, J. C., Hodgman, C. E. 2015, “Methods for activating natural energy metabolism for improved yeast cell-free protein synthesis,” U.S. Patent Application Ser. No.: 62/098,578. See also Guarino, C., & DeLisa, M. P. (2012). A prokaryote-based cell-free translation system that efficiently synthesizes glycoproteins. Glycobiology, 22(5), 596-601. The contents of all of these references are incorporated in the present application by reference in their entireties.

In some embodiments, the method includes adding one or more components to the unclarified lysate to prepare a “CFPS reaction mixture”. The term “reaction mixture,” as used herein, refers to a solution containing reagents necessary to carry out a given reaction. This mixture typically contains the unclarified lysate, an RNA translation template, and a suitable reaction buffer for promoting cell-free protein synthesis from the RNA translation template. The RNA translation template can be an exogenous RNA translation template. In some aspects, the CFPS reaction mixture includes a DNA expression template encoding an open reading frame operably linked to a promoter element for a DNA-dependent RNA polymerase or a DNA-dependent RNA polymerase to direct transcription of an RNA translation template encoding the open reading frame. In these other aspects, additional nucleotides (NMPs, NDPs, or NTPs) and divalent cation cofactor(s) can be included in the CFPS reaction mixture.

The reaction buffer can include any organic anion suitable for CFPS (e.g., glutamate, acetate, among others). In certain aspects, the concentration for the organic anions is independently in the general range from about 0 mM to about 300 mM, including intermediate specific values within this general range, such as about 0 mM, about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM and about 300 mM, for example.

The reaction buffer can include any halide anion suitable for CFPS. Generally, the concentration of halide anions, if present in the reaction, is within the general range from about 0 mM to about 300 mM, including intermediate specific values within this general range, such as those disclosed for organic anions generally herein.

The reaction buffer can include any polyether suitable for CFPS. In certain aspects, the polyether can be a polymer of ethylene oxide, such as molecular weight variants of polyethylene glycol (PEG), among others. In certain aspects, the concentration of polyethers in the reaction can be in the general range from about 0% to about 10% (w/v), about 0.5% to about 6% (w/v), about 1% to about 5% (w/v), or about 2% to about 4% (w/v). In some cases, more than one polyether is present.

The reaction buffer may include any organic cation suitable for CFPS. In certain aspects, the organic cation can be a polyamine, such as spermidine or putrescine, among others. Preferably polyamines are present in the CFPS reaction. In certain aspects, the concentration of organic cations in the reaction can be in the general range from about 0 mM to about 3 mM, about 0.5 mM to about 2.5 mM, or about 1 mM to about 2 mM. In some cases, more than one organic cation is present.

The reaction buffer may include any inorganic cation suitable for CFPS. Suitable inorganic cations can include monovalent cations, such as sodium, potassium, lithium, among others; and divalent cations, such as magnesium, calcium, manganese, among others. In some cases, the inorganic cation is magnesium. The magnesium concentration can be within the general range from about 1 mM to about 50 mM, including intermediate specific values within this general range, such as about 1 mM, about 2 mM, about 3 mM, about 5 mM, about 6 mM, about 7 mM, about 8 mM, about 9 mM, about 10 mM, about 11 mM, about 12 mM, about 13 mM, and about 14 mM, for example.

The reaction mixture may further include endogenous nucleotides (i.e., NTPs that are present in the lysate) and or exogenous nucleotides (i.e., NTPs or NMPs that are added to the reaction mixture). In some cases, the reaction uses ATP, GTP, CTP, and UTP. The concentration of individual NTPs can be within the range from about 0.1 mM to about 5 mM. Rather than or in addition to NTPs, the reaction mixture may include endogenous or exogenous NMPs such as AMP, GMP, CMP, and UMP within the range from about 0.1 mM to about 5 mM.

The reaction mixture may further include endogenous amino acids (i.e., amino acids that are present in the cell extract) and or exogenous amino acids (i.e., amino acids that are added to the reaction mixture). In some cases, the reaction uses all 20 canonical amino acids. In some cases the reaction uses non-canonical amino acids. The concentration of individual amino acids can be within the range from about 0.1 mM to about 5 mM.

The reaction mixture may further include phosphorylated or non-phosphorylated energy sources. Non-limiting examples include phosphoenolpyruvate, glutamate, glycerol, pyruvate, glucose, and creatine phosphate.

The reaction mixture may further include endogenous tRNAs (i.e., tRNAs that are present in the cell extract) and or exogenous tRNAs (i.e., tRNAs that are added to the reaction mixture). The concentration of tRNAs can be within the range from about 10 μg/mL to about 1 mg/mL.

The reaction mixture may further include chemicals to modulate the oxidation-reduction potential of the extract. These chemicals may include oxidized or reduced glutathione at concentrations of 0.1 to 30 mM or a sulfhydryl inactivating agent, such as iodoacetamide at concentrations of 1 to 5000 μM.

The reaction mixture may additionally contain enzyme cofactors and metabolites, such as folinic acid, nicotinamide adenine dinucleotide, coenzyme-A, or sodium oxalate.

The reaction mixture may also any alcohol suitable for CFPS. In certain aspects, the alcohol may be a polyol. In certain aspects the alcohol is between the general range from about 0% (v/v) to about 25% (v/v), including specific intermediate values of about 5% (v/v), about 10% (v/v) and about 15% (v/v), and about 20% (v/v), among others.

The reaction mixture may include an expression template, a translation template, or both an expression template and a translation template. The reaction mixture may comprise one or more polymerases capable of generating a translation template from an expression template. The polymerase may be supplied exogenously or may be supplied from the organism used to prepare the lysate. In certain specific embodiments, the polymerase is expressed from a plasmid present in the organism used to prepare the lysate and/or an integration site in the genome of the organism used to prepare the lysate.

The protein synthesis can be performed at any temperature suitable for CFPS. Temperature may be in the general range from about 10° C. to about 42° C., including intermediate specific ranges within this general range, including from about 15° C. to about 37° C., from about 15° C. to about 30° C., and from about 15° C. to about 25° C. In certain aspects, the reaction temperature can be about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C.

### B. Unclarified Lysates and Kits for CFPS

The unclarified lysate prepared according to one or more of the embodiments above can be characterized by several properties. For example, the activity of the unclarified lysate can be characterized by that volumetric yield for batch reactions producing a reporter protein (e.g., GFP or luciferase).

Compared with clarified extracts, the unclarified lysate of the present disclosure may provide for more efficient protein synthesis, e.g., due to the presence of endogenous cellular structures (e.g., microsomes), which provide glycosyltransferases for glycoprotein synthesis, chaperones, and other molecules necessary for disulfide bridge formation that would be removed by clarification steps, in addition to time, energy and equipment costs savings.

In some embodiments, the unclarified lysate extract of the present disclosure is part of a “CFPS reaction mixture”, as described above. A reaction mixture is referred to as complete if it contains all reagents necessary to perform the reaction. Components for a reaction mixture may be stored separately in separate containers, each containing one or more of the total components. Components may be packaged separately for commercialization and useful commercial kits may contain one or more of the reaction components for a reaction mixture. Thus, the unclarified lysate of the present disclosure can be separately packaged as a component of a CFPS reaction mixture kit.

### C. Systems

Embodiments of the present disclosure include a system for CFPS using an unclarified lysate. The system can operate in a batch, continuous, or semi-continuous manner. The system can be enclosed in a closed sterile environment, so as to perform the entire process in a sterile manner.

The system can include at least one lysis vessel adapted to lyse cells of a cell culture. The lysis vessel can be in fluid communication with a reaction vessel for CFPS. A lysis vessel can be any container suitable for the selected method of lysis (e.g., adapted for lysing cells by freezing and thawing, or adapted for lysing cells by homogenization). The vessel can be selected based on the volume of the cell culture, the harvested cells, or the resuspended cells. Examples of suitable vessels include, but are not limited to, a tank, a test tube, a beaker, a flask, or a multi-well plate.

In some cases, the system further includes the bioreactor containing the starting material, e.g., cultured cells. The bioreactor can be any type of bioreactor like a batch or a fed batch bioreactor or a continuous bioreactor like a continuous perfusion fermentation bioreactor. The bioreactor can be made of any suitable material and can be of any size. Typical materials are stainless steel or plastic. In a particular embodiment, the bioreactor is a disposable bioreactor, e.g., in form of a flexible, collapsible bag, designed for single-use. Lysis can be performed directly in the bioreactor. For example, the system can include a shear device to disrupt the cellular membrane to obtain the cell lysate. Alternatively, the bioreactor can be used for culturing the cells and lysis can be performed in a different vessel. In another example, fluid can be recirculated from the bioreactor to an external piece of lysis equipment such as a homogenizer, for example.

In some embodiments, the system includes a device configured to concentrate the cell culture. For example, the concentrating device can be a centrifuge, tangential flow filtration device, hollow-fiber filtration apparatus, or a membrane separation device such as a woven or non-woven membrane, an electrospun membrane, a hollow fiber membrane, rolled sheet membrane, or flat sheet membrane.

The system can include a reservoir that contains a resuspension medium configured to supply the medium to the lysis vessel, a device for resuspending the cells in the lysis vessel (e.g., a mixing device), and/or a device for lysing the cells (e.g., a sonicator, homogenizer, nitrogen cavitation device, heat exchanger, bead-beating system, freeze-thaw device, a syringe, or other device configured to disrupt cell membranes).

In some cases, the system includes post-lysate devices to further treat the extract. For example, the system can be configured or adapted to agitate or shake the lysate, to include apparatuses to cool or heat the lysate (as in a “run-off” reaction), or ports for delivering chemicals including chelators, oxidation-reduction-modifying chemicals such as GSSG, GSH, and iodoacetamide.

The system can be configured to deliver the unclarified lysate to a reactor vessel for cell-free protein synthesis. Alternatively, the system can be configured for freezing or freeze-drying the unclarified lysate and for storing the unclarified lysate (e.g., in liquid nitrogen for storage at −80° C., −20° C., or 4° C.).

Each device used in a system typically employs a process equipment unit, that includes the pumps (e.g., vacuum or peristaltic), valves, sensors and device holders for controlling flow of the lysate. In some embodiments, the system is configured for continuous fluid communication between the various devices (i.e., that the fluid flows directly through all the devices without interruptions). In other embodiments, one or more valves, sensors, detectors, surge tanks and equipment for any in-line solution changes may be included between the various devices, thereby to temporarily interrupt the flow of fluid through the system, if necessary, for example, to replace/remove a particular unit operation.

In some embodiments, the system further includes one or more sensors and/or probes for controlling and/or monitoring one or more process parameters inside the system, for example, temperature, pressure, pH, conductivity, dissolved oxygen (DO), dissolved carbon dioxide, mixing rate, flow rate, or other product parameters. The sensor may also be an optical sensor in some cases. Process control may be achieved in ways which do not compromise the sterility of the system. Sensors and/or probes may be connected to a sensor electronics module, the output of which can be sent to a terminal board and/or a relay box. The results of the sensing operations may be input into a computer-implemented control system (e.g., a computer) for calculation and control of various parameters (e.g., temperature, weight/volume measurements, and purity) and for display and user interface. Such a control system may also include a combination of electronic, mechanical, and/or pneumatic systems to control process parameters. It should be appreciated that the control system may perform other functions and the invention is not limited to having any particular function or set of functions.

The following Examples are intended to illustrate the above invention and should not be construed as to narrow its scope. Numerous variations and modifications may be made while remaining within the scope of the invention.

### EXAMPLES

### CFPS Using Unclarified Cellular Lysate

Reported methods for CFPS extract production to date require at least centrifugation for lysate clarification during the post-lysis processing step. In particular, the removal of cell wall fragments and genomic DNA via centrifugation has been viewed as critical for extract activity.

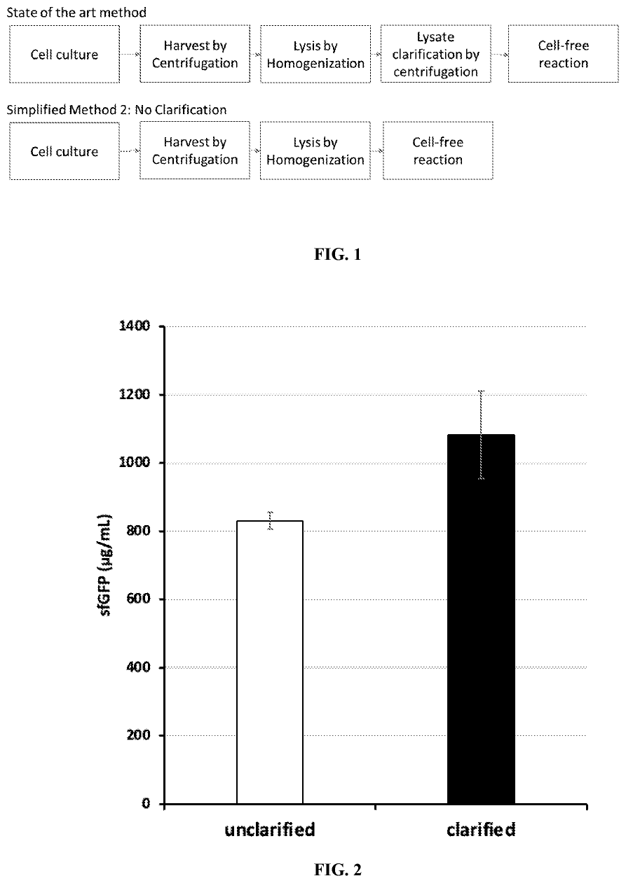

Against these teachings, the present examples provide methods for CFPS using unclarified lysate (FIG. 1). The following results show substantial yields from the cell-free reaction, while reducing the time and equipment costs associated with cell-free protein synthesis. For example, the operation time for clarifying a cellular lysate using a centrifuge can range from about 30 minutes to up to 24 hr. Additional time is required to clean the centrifugation equipment (e.g., up to 48 hr of cleaning time at scale). Centrifugation equipment can cost $10,000-$1,000,000, to purchase and have annual operating costs of $50,000-$200,000, depending on scale. Indeed, at certain scales, a process that removes the need for this additional centrifugation during extract preparation may produce protein at a lower cost or time per gram even if the titer of protein produced in the reaction using this extract is slightly lower. This process may also be able to be implemented in more resource-limited or distributed manufacturing settings.

Cells were cultured, harvested, and lysed according to conventional methods. The control extracts were clarified by centrifugation to produce an E. coli extract as previously reported in S30 E. coli extract preparation protocols.

As shown in FIG. 2, unclarified lysates provided a substantial percentage of protein yields (here monitored by synthesis of superfolder green fluorescent protein) compared to the state-of-the-art centrifugation method.

Other embodiments of the present disclosure are possible. Specific examples should not be construed as limiting the scope of the disclosure, but as merely providing illustrations of embodiments of this disclosure. Various combinations or sub-combinations of the specific features and aspects of the embodiments may be made.

The scope of the present disclosure is accordingly to be limited only by the appended claims. The reference to an element in the singular is not intended to mean “one and only one” unless explicitly so stated, but rather “one or more.” All structural, chemical, and functional equivalents to the elements of the above-described Example that are known to those of ordinary skill in the art are encompassed by the present claims. Furthermore, no element, component, or method step in the present disclosure is intended to be dedicated to the public regardless of whether the element, component, or method step is explicitly recited in the claims.

Various examples have been described. These and other examples are within the scope of the following claims.

What is claimed is:

1. A method of improving bioprocessing efficiency of cell-free protein synthesis comprising:
preparing a cellular lysate, wherein the prepared cellular lysate has not been clarified,
contacting the prepared cellular lysate with a nucleic acid template encoding a polypeptide, and
optionally isolating the polypeptide encoded by the nucleic acid template.

2. The method of claim 1, wherein preparing the cellular lysate comprises harvesting cells of a cellular culture and lysing the cells.

3. The method of claim 2, wherein the cells are harvested by subjecting a cellular culture to centrifugation, tangential flow filtration, membrane separation or a combination thereof, optionally membrane separation including hollow fiber membrane separation.

4. The method of claim 2, wherein the harvested cells are lysed by sonication, homogenization, nitrogen cavitation, freeze-thaw, syringing, chemical, enzymatic, or osmotic lysis.

5. The method of any one of claims 2-4, wherein the harvested cells are lysed by homogenization at an operating pressure within a range of about 2000 to 30,000 psig, optionally about 5000 to about 30,000 psig.

6. The method of any one of claims 2-4, wherein harvested cells are lysed at a temperature within a range of about 1° C. to about 30° C., about 1° C. to about 25° C., about 1° C. to about 20° C., or about 1° C. to about 15° C.

7. The method of any one of claims 1-6, wherein contacting the prepared cellular lysate with the nucleic acid template is performed at a temperature of about 10° C. to about 42° C., about 15° C. to about 37° C., from about 15° C. to about 30° C., and from about 15° C. to about 25° C.

8. The method of any one of claims 1-7, wherein the cellular lysate comprises lysed cells from a transgenic cell culture, a mammalian cell culture, a bacterial cell culture, plant cell culture, a yeast cell culture, an insect cell culture, a fungal cell culture, or an algal cell culture.

9. The method of any one of claims 1-8, wherein the cellular lysate comprises lysed cells from an E. coli culture.

10. The method of any one of claims 1-9, wherein the cellular lysate is prepared from a cell culture that is in log or stationary phase.

11. The method of any one of claims 1-10, further comprising a step of culturing cells to an OD₆₀₀ of about 0.5 to about 100, optionally about 5 to about 100 or about 10 to about 100.

12. The method of any one of claims 1-11, further comprising mixing the cellular lysate with one or more components selected from the group consisting of amino acids, nucleotides, salts, cofactors, an energy source, a translation template, and a transcription template, or a combination thereof.

13. The method of claim 12, wherein the energy source comprises a phosphate group or non-phosphorylated energy group.

14. The method of claim 12, wherein the energy source is present at a concentration of greater than about 10 mM, 100 mM, 200 mM, 300 mM.

15. The method of claim 14, wherein the energy source includes at least one of phosphoenolpyruvate, glutamate, glycerol, pyruvate, glucose, or creatine phosphate.

16. The method of any one of claims 12-15, wherein the salt includes potassium at a concentration greater than about 50 mM, 100 mM, 150 mM, 200 mM, 250 mM, 300 mM, 350 mM, 400 mM, or 450 mM, but less than about 500 mM, magnesium at a concentration greater than about 1 mM, 2 mM, 3 mM, 4 mM, 5 mM, 6 mM, 8 mM, 12 mM, 16 mM or 20 mM, but less than about 30 mM, or a combination thereof.

17. The method of any one of claims 1-16, further comprising freeze-drying the cellular lysate.

18. The method of any one of claims 1-17, further comprising freezing the cellular lysate.

19. The method of any one of claims 1-18, comprising isolating the polypeptide encoded by the nucleic acid template.

20. The method of claim 19, wherein isolating the polypeptide is performed at a temperature within a range of about 1° C. to about 30° C., about 1° C. to about 25° C., about 1° C. to about 20° C., or about 1° C. to about 15° C.

21. A system for continuous bioprocessing for cell-free protein synthesis comprising:
a lysing device configured to prepare a cell lysate from cultured cells, wherein the lysing device in fluidic connection with
a reaction chamber configured for cell-free protein synthesis, wherein the reaction chamber is configured to receive the prepared cellular lysate and a nucleic acid template encoding a polypeptide, and
optionally further comprising a bioreactor for culturing cells;
optionally wherein the bioreactor is operably connected to a cell concentrating device for concentrating the cultured cells
optionally wherein the cell concentrating device is operably connected to the lysing device.
